(19) United States
(12) Patent Application Publication
(10) Pub. No.: US 2018/0252722 A1
(43) Pub. Date: Sep. 6, 2018

(54) PCR-based Method for Counting Circulating Tumor Cells

(71) Applicant: Yan Wang, San Diego, CA (US)

(72) Inventor: Yan Wang, San Diego, CA (US)

(21) Appl. No.: 15/904,393

(22) Filed: Feb. 25, 2018

Related U.S. Application Data
(60) Provisional application No. 62/467,181, filed on Mar. 5, 2017.

(51) Int. Cl. G01N 33/574; C12N 15/10; C12Q 1/686

(52) U.S. Cl. CPC G01N 33/57492; C12N 5/0634; C12Q 1/686; C12N 15/1096

(57) ABSTRACT

Disclosed are PCR-based methods for enumerating circulating tumor cells in a blood or lymphatic fluid sample. The cell sample may or may not be enriched for the circulating tumor cells. The cell sample is first fractionated into many fractions such that each fraction practically contains no more than one circulating tumor cells. RNA and DNA are extracted from cells in each fraction, RNA transcripts are converted to cDNA using a reverse transcriptase, and PCR amplification is performed in each fraction for detection of tumor-specific sequences. The number of circulating tumor cells in the sample is counted as the number of fractions having cells that contain tumor-specific sequences.

### CROSS-REFERENCE TO RELATED APPLICATIONS

This application claims the benefit of U.S. provisional patent application No. 62/467,181, filed Mar. 5, 2017, the content of which is incorporated by reference herein.

### FIELD OF THE INVENTION

This invention relates to methods and technologies in the field of molecular oncology, especially relates to methods for detecting and counting circulating tumor cells in blood and lymph samples using a highly sensitive PCR-based method.

### BACKGROUND OF THE INVENTION

Circulating tumor cells (CTCs) are tumor cells shed into the bloodstream from primary and metastatic tumor tissues. Compared to the invasive tumor tissue biopsy, the CTCs can serve as a “real time liquid biopsy” that is non-invasive, easy to obtain, and can be used to monitor the real time tumor progression. The isolation and analysis of CTCs thus hold great promise for the early detection of invasive cancers, the prognostic prediction of cancer therapy, the detection of drug-resistant profiling, and the management of advanced diseases. Although CTC analysis has great potential for clinical applications, the rarity of CTCs in blood samples (e.g. 1-10 per ml) and the heterogeneity of CTC population have posed a great technological challenge for detection and analysis of these cells.

There are about 8×10⁹ red blood cells and 5×10⁶ white blood cells as compared to 1-10 CTCs in 1 ml blood sample. Searching for the CTCs in the sea of blood cells is analogous to “finding the needle in the haystack”. To make things even worse is the heterogeneous nature of CTCs that make it hard to find general genetic or cytological markers for enriching and detecting the CTCs. The most commonly used marker for enriching CTCs is the cell surface protein epithelial cell adhesion molecule (EpCam), which is expressed in CTCs of epithelial sources. Using EpCam-based immunomagnetic approaches to enrich CTCs will lead to loss of CTCs that do not express or express low levels of EpCam. To circumvent this problem, multiple antibodies that target to different tumor-specific antigens are used to select CTCs. Another method to select CTCs is based on size differences among different cell types as tumor cells generally have larger sizes than those of white/red blood cells. However, since the sizes of tumor cells vary among different types of tumor cells, small tumor cells may have a similar size as white blood cells. Isolation methods based on size differences can result in loss of small tumor cells. The loss of CTCs during the enrichment process is one factor contributing to the inaccuracy of CTC detection and enumeration.

The current CTC detection methods can be divided into two major types: nucleic acid-based and cytometric-based approaches. The nucleic acid-based detection method uses polymerase chain reaction (PCR) for detection of DNA or RNA sequences that are differentially expressed by tumor cells. With the use of multiple tumor-specific markers, this PCR-based method can be a highly sensitive one. It can be performed without enrichment, but at expense of sensitivity. This method detects nucleic acids prepared from lysed cells, which is unable to visualize cells for morphology and enumeration. Another disadvantage is that it is difficult to standardize techniques across laboratories, making it difficult for reliable use in clinical settings. The cytometric-based detection method uses immunohistochemistry and immunofluorescence to visualize the cell morphology and enumerate CTCs. The cells from blood samples are first pretreated to enrich the tumor cells. The enriched cells are then visualized by staining with antibodies to multiple tumor-specific markers and optionally a white blood cell-specific marker. The cells with positive stains for at least one tumor-specific marker and negative stain of the white blood cell-specific marker is identified as a tumor cell. In this way, this method offers high specificity in detecting CTCs. However, this method depends on the expression level of the tumor-specific markers in the tumor cells and the specificity of antibodies used. Since no markers are purely specific for tumor cells and marker expression levels vary among different tumor cells, false-positive and false-negative detection is a likely outcome. The sensitivity of this method is largely dependent on the specificity and binding strength of the antibody for the tumor-specific markers. In general, this method offers a higher specificity but has a lower sensitivity than the nucleic acid-based method.

Therefore, there is an urgent need for developing CTC detection technologies that offer both high sensitivity and high specificity, and can produce comparable results across laboratories. It will be ideal that the detection method can be applied to less processed or even nonenriched blood samples to minimize the loss of CTCs. The present invention offers such advantages and provides other benefits as well.

### SUMMARY OF THE INVENTION

The current PCR-based method for CTC detection offers a sensitive approach to detect tumor cells escaped into the circulating blood or lymph. A disadvantage of existing PCR-based detection method is that it requires lysing the cells before the PCR analysis can be performed, making it unable to enumerate the CTCs. The present invention provides a PCR-based method for CTC enumeration by first fractionating a cell sample into many fractions before nucleic acid extraction and PCR analysis. The sample containing CTCs is fractionated to an extent such that more than 50% of the fractions contain no more than one circulating tumor cell per fraction. Many of the factions will not contain any CTC, and for fractions containing the CTCs, most of them will just contain a single one. DNA and RNA are extracted from cells in each fraction. A PCR amplification and detection is performed to identify fractions with cells that express tumor-specific genes higher than the background level. Such cells are identified as circulating tumor cells.

The present invention provides a method for counting circulating tumor cells in a sample, comprising the steps of: a) fractionating the sample into a plurality of equal fractions, wherein more than 50% of the fractions contain no more than one circulating tumor cell per fraction; b) extracting RNA and/or DNA from cells in each fraction and generating cDNA from the extracted RNA; c) performing amplification reactions in each fraction using at least one pair of tumor-specific primers for amplification of tumor-specific sequences; d) detecting amplified tumor-specific sequences in each fraction; and e) counting the number of fractions with amplified tumor-specific sequences to determine the number of CTCs in the sample. In some embodiment, each fraction contains no more than one single CTC.

This method can be used to detect CTCs from any cell sample suspected of containing tumor cells, for example, a patient's blood sample and a patient's lymphatic fluid sample. The sample can be enriched for CTCs by removing non-tumor cells (e.g. red and white blood cells) or can be used in a nonenriched format. The sample may be pretreated to dissociate cell clusters if any to ensure cells are separated into single individual cells in the sample. The sample is fractionated into many equal volume fractions such that no single fraction is likely to have more than one CTC. Every fraction contains no more than one CTC but may contain many non-tumor cells (e.g. white blood cells). The sample can be fractionated into, for example, more than 10, 100, 1000, 10,000 and 100,000 fractions. The method for fractionating cells into many compartments include, for example, directly adding them into multi-well PCR plates, using microfluidic chips (e.g. digital PCR chip) to distribute them into thousands of compartments, or encapsulating them into thousands of microdroplets using a droplet generator.

In some embodiment, the sample is enriched for CTCs by removing non-tumor cells such as red blood cells, white blood cells and lymphocytes. CTCs can be enriched on the basis of the size difference between tumor cells and blood cells. The large CTCs are selected and enriched while the white blood cells of smaller size are removed. The sample can also be enriched for CTCs by selecting cells binding to antibodies against selective cell surface antigens such as EpCam. A CTC enriched sample can be fractionated to such an extent that each fraction practically contains no more than one single cell per fraction. In this format, a fraction may contain no cells, one CTC or one non-tumor cell. The tumor gene expression comparison is made between a circulating tumor cell and a non-tumor cell, allowing smaller difference in gene expression to be detected. The identified single CTC can be retrieved and used for further analysis of its genotype and expression profiling.

Selecting the right tumor-specific marker genes is critical for successful detection and identification of CTCs in a sample. If the sample is collected from a patient with known cancer type(s), the tumor-specific genes can be selected to be cancer genes specific for the known cancer type(s). It is preferable to choose cancer genes only expressed in the cancer cells, but not in normal blood cells. Using these cancer-only marker genes allow detection of CTCs against the background of thousands and millions of non-tumor cells in minimally processed or nonenriched cell samples. Cancer-only marker genes can be found by comparing cancer cell expression profiles with those of normal blood cells. Cancer specific marker gene sets can be selected for specific cancer types, for example, breast cancer, lung cancer, liver cancer, pancreatic cancer, prostate cancer, stomach cancer, kidney cancer, intestinal cancer, colon cancer and melanoma. One or more cancer-specific marker gene sets can be used to detect CTCs originated from a single or multiple cancer types. For detection of CTCs from an unknown cancer source, a spectrum of cancer marker genes from different cancer types or pan-cancer marker genes can be used. In some embodiment, the tumor-specific marker genes are selected from cancer genes related to metastasis. Identification of circulating tumor cells that express metastasis related genes can be of clinical importance because this subset of CTCs might be the seed cells that can grow more metastatic tumors.

The CTC-enriched cell preparation, RT-PCR reagents, and reporter probes are mixed together and partitioned into many compartments as described above. The CTC-enriched cells used for the partitioning may be live or fixed cells, wherein DNA and RNA shall be maintained within the cells. The RT-PCR regents include a PCR buffer, dNTPs, thermostable DNA polymerases, tumor-specific primers, thermostable reverse transcriptases, and RNAse inhibitors. Cells in each fraction are lysed by heat in the PCR buffer and cDNA molecules are generated from the released RNA using a reverse transcriptase. The tumor-specific DNA sequences in CTCs are amplified by PCR using tumor-specific primer pairs. In some embodiment, the tumor-specific DNA sequences are tumor-specific cDNA converted from RNA transcripts. In some embodiment, the tumor-specific DNA sequences are genomic DNAs, for example, cancer-related fusion genes and cancer-related genes that undergo copy number multiplication in tumor cells. The method can be used to detect CTCs containing tumor-specific RNA transcripts, tumor-specific genomic DNA, or both.

The amplified tumor-specific sequences can be detected by any conventional methods for detecting PCR products. The PCR products can be detected at the end of the PCR or during the PCR cycles. In some embodiment, the amplified tumor-specific sequences are detected by a real time quantitative PCR using a non-specific dsDNA binding dye or a sequence-specific reporter probe. The detection method using sequence-specific reporter probes is preferred because of its high specificity. In addition, different cancer genes or cancer genes related to specific cancer types can be detected by use of different reporter probes. In some embodiment, the amplified tumor-specific sequences are detected by a digital PCR.

In some embodiment, the present invention provides a method of detecting the presence of cancer in a subject, comprising a) determining the presence of CTCs in a blood sample or a lymphatic fluid sample from the subject using the method of the present invention; and b) using the presence of CTCs as indicative of the presence of cancer in the subject.

In some embodiment, the present invention provides a method of making prognostic prediction of a cancer treatment in a patient, comprising a) counting CTCs in a patient's blood sample or lymphatic fluid sample at different times during or after the cancer treatment using the method of the present invention; and b) using a increasing and decreasing trend of CTC count in patient's sample as indicative of a poor and good prognosis, respectively. In some embodiment, the CTCs are tumor cells that express metastasis-related cancer genes.

In some embodiment, the present invention provides a method of detecting cancer metastasis in a patient, comprising a) detecting the presence of CTCs with expression of metastasis-related cancer genes in patient's blood sample or lymphatic fluid sample using the method of the present invention; and b) using the presence of CTCs with expression of metastasis-related cancer genes as indicative of the presence of cancer metastasis in the patient.

### DETAILED DESCRIPTION

### Definitions

Unless otherwise defined, all technical and scientific terms used herein have the same meaning as commonly understood by one of the ordinary skill in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The term “tumor specific sequences”, as used herein, refers to nucleic acid sequences including RNA or DNA sequences that have higher representation in cancerous cells as compared to normal cells, especially normal blood cells or lymphatic cells. Tumor specific RNA sequences may be RNA transcripts that are expressed in one or more types of cancer cells, but have very low or no expression in normal blood or lymphatic cells. The tumor specific genes that are only expressed in tumor cells with no detectable expression in normal blood or lymphatic cells are referred as “cancer-only marker genes”. Tumor specific DNA sequences are DNA sequences that are only present in cancer cells or over-represented in cancer cells, including, for example, cancer-related gene fusion sequences and gene sequences with copy number multiplication in cancer cells. The tumor specific sequences may be over-represented in a specific cancer type or many different cancer types. The tumor specific sequences related to a specific cancer type (e.g. breast cancer) can be selected as a cancer type specific marker sequence set and used to detect tumor cells related to the particular cancer type. The tumor-specific sequences that are expressed in many different cancer types can be used as pan-cancer markers for detection of cancer occurrence.

The term “tumor specific primers”, as used herein, refers to PCR primers that are designed to amplify tumor specific sequences in polymerase chain reactions. The tumor specific primers are designed such that they specifically amplify respective tumor specific sequences, but not other sequences present in normal cells. For example, the tumor specific primers for amplification of tumor specific RNA transcripts are designed to produce amplicons spanning multiple exons so that they will not amplify genomic sequences of the corresponding genes. The tumor specific primers for amplification of a gene fusion sequence are designed to produce an amplicon across the fusion junction so that the gene sequence to be amplified exists only in cancer cells.

The term “circulating tumor cells”, as used herein, refers to tumor cells that are dissociated from the original tumor, enter into the vasculature or lymphatic system and are carried around the body by circulation. These cells carry tumor cell specific expression profiles and tumor-specific genotype and can become the seed cells to grow into metastatic tumors. The circulating tumor cells can be identified by their expression of tumor specific genes and lack of expression of blood cell specific markers such as CD45.

The term “a sequence-specific reporter probe”, as used herein, refers to a reporter probe that comprises a signature nucleotide sequence and a reporter moiety, which can give off a detectable signal upon binding to its complementary sequence. A preferable detectable signal is a fluorescent signal. By coupling to different fluorophores, different sequence-specific reporting probes can be used in the same reaction for multiplexed detection of multiple target sequences. On the other hand, reporter probes with different signature nucleotide sequences and the same fluorophore can be used to determine the total amount of a plurality of target sequences. There are many sequence-specific reporter probes that can be used in the present invention, including, but not limited to, 5′-nuclease Taqman® probes Scorpion® probes and Molecular beacons probes, light up probes, and adjacent probes (Marras SAE, et al. Clinica Chimica Acta 2006, 363:48-60).

The present invention provides a method for counting circulating tumor cells in blood or lymph samples using a highly sensitive PCR-based detection method. The existing CTC detection methods are generally divided into two categories: PCR-based methods that offer high sensitivity but lacks the capability of direct enumeration of CTCs; and cyotmetric-based methods that can enumerate CTCs but have relatively low sensitivity. Both methods usually require significant enrichment of CTCs, a process by itself that may lead to loss of CTCs. The present invention first fractionates a cell sample suspected of containing CTCs into many fractions such that no fraction is likely to have more than one CTC and then identifies CTCs as cells that express at least one of selective tumor specific marker genes at a higher level than the baseline level in other non-tumor cells. The present invention combines the benefit of high sensitivity of a PCR-based detection method with the capability of direct enumeration of individual CTCs. The sensitivity of this method comes from the use of PCR that can greatly amplify the tumor specific signals from CTCs. By use of a spectrum of tumor specific marker genes, the present invention is likely to detect more CTCs known for their heterogeneity than other methods using only one or a few biomarkers. The tumor specific marker genes can selected based on cancer types or cancer stages, thus providing richer information about individual CTCs. With the capability of comparing the gene expression of a single CTC with a non-tumor cell, the present invention enables the identification of CTCs that will be escaped from the radar of other detection methods. In another aspect of the present invention, the identified individual CTCs can be retrieved and subjected to further genotyping and gene expression profiling analysis. Yet as another benefit of this invention, it can be applied to minimally processed or non-enriched cell samples without compromise in detection sensitivity when cancer-only marker genes are used for detecting CTCs.

The method of the present invention for detecting CTCs in a sample comprises the steps of: a) fractionating the sample into a plurality of equal fractions, wherein more than 50% of the fractions contain no more than one circulating tumor cell per fraction; b) extracting RNA and DNA from cells in each fraction and generating cDNA from the extracted RNA; c) performing amplification reactions in each fraction using at least one pair of tumor-specific primers for amplification of tumor-specific sequences; d) detecting amplified tumor-specific sequences in each fraction; and e) counting the number of fractions with amplified tumor-specific sequences to determine the number of CTCs in the sample.

This method is designed to detect tumor cells in body fluid such as blood or lymph by detecting tumor specific gene expression or genotype in a single tumor cell. The cell sample used for the present invention can be blood or lymph sample collected from a subject. CTCs are usually enriched from the original blood samples before subjected to detection method. For example, red blood cells can be easily lysed and removed from the sample. Magnetic beads coupled with epithelial cell surface marker EpCam antibodies are used to select a cell population enriched with CTCs, or white blood cells can be removed from the sample using anti-CD45 and/or anti-CD66 magnetic beads. CTCs can also be selected based on size differences between CTCs and white blood cells using a filtration method. A microfluidic CTC-iChip designed to deplete red blood cells, platelets and white blood cells from the blood sample can be used in the present invention to enrich both EpCam⁺ and EmCam⁻ CTCs (Emre Ozkumur et al. Science Translational Medicine 5, 179ra47 (2013)). The CTC-iChip method first debulks red blood cells and platelets from nucleated cells including CTCs and white blood cells, and then purifies CTCs by deleting white blood cells tagged with anti-CD45 and anti-CD15 antibodies. This negative selection method is preferable because it allows capturing of CTCs independent of their cell sizes and expression of surface antigens. Similar methods can also be applied to purify CTCs from lymph samples to obtain a CTC-enriched cell preparation.

The CTC-enriched cell preparation is fractionated into many tiny compartmentalized fractions such that more than 50% of fractions do not contain more than one CTC per fraction. Before cell fractionation, the cell preparation may need to be dissociated into single cells using mild enzyme digestion or may need to be fixed to prevent RNA and DNA loss due to cell damage or cell lysis during the processing procedure. The fixative reagents can be, for example, ethanol, paraformaldehyde, formalin, or formamide. It is very important to maintain RNA and DNA within the cells before they are fractionated into the tiny compartmentalized fractions. There are two operational formats for this fractionation. For the first format, the cell sample are fractionated such that each faction has practically no more than one single cell per fraction. For the second format, the cell sample are fractionated such that each faction has practically no more than one single CTC per fraction, but may have more than one non-tumor cells. The cell preparation can fractionated into more than 10, 100, 1000, 10,000, 100,000 or 1,000,000 fractions. The methods to partition cells into many compartments are well known in the art. For example, cells can be directly added into multi-well PCR plates (e.g. 96-well plate or 384-well plate). Using microfluidic chips (e.g. digital PCR chip), cells can be distributed into thousands of tiny compartments. Cells can also be fractionated and encapsulating into microdroplets using a droplet generator. During the fractionation, CTC-enriched cells, RT-PCR reagents, and reporter probes are mixed together and equally added to each fraction. The RT-PCR reagents include a PCR buffer, ATP, dNTPs, thermostable DNA polymerases, tumor-specific primers, thermostable reverse transcriptases, and RNAse inhibitors.

Cells in each fraction are lysed by heat in the PCR buffer. For example, the cells can be heated at 95-99° C. for 10-20 minutes to break cell membranes and release nucleic acids in the cells. Surfactants such as NP-40, Tween-20, TritonX-100 can also be added to aid in breaking cell membranes and completely releasing DNA and RNA. The DNA and RNA released from the lysed cells can be directly used for downstream reverse transcription and PCR amplification. In some embodiment, only tumor-specific RNA transcripts are converted to cDNA using tumor-specific primers. The tumor-specific cDNA can be then amplified and detected using the same primers in subsequent PCR. Alternatively, all the RNA transcripts can be converted to cDNA using random primers.

The present invention exploits the tumor-specific RNA or DNA marker sequences to identify rare CTCs surrounded by many non-tumor cells such as blood cells or lymphocytes. Selecting tumor-specific sequences having low or no representation in the background non-tumor cells is essential for the successful detection of CTCs. The preferable tumor-specific sequences are cancer-only DNA or RNA sequences that have no representation in the non-tumor cells, for example, somatic gene mutations accumulated in tumor cells but not present in normal cells or RNA transcripts not expressed in normal blood cells. Cancer-only RNA transcripts that are specific for a particular cancer type or have pan-cancer expression are reported by Harber et al. (WO/US2016/154600). Harber et al. first search tumor-specific gene candidates by comparing cancer cell expression profiles with those of normal blood cells. The expression of the tumor-specific candidate genes in cancer cells and normal blood cells are experimentally determined by use of PCR amplification. The tumor-specific genes that truly have no expression in blood cells are selected as cancer-only marker sequences. Tumor-specific primers are designed to span multiple exons of the target transcripts so as to prevent amplification of genomic sequence of the corresponding gene. By detecting these cancer-only marker gene, a single CTC can be detected in the context of hundreds, thousands, even millions of blood cells. In one embodiment, CTC-containing cells are fractionated such that each fraction practically contains no more than one CTC, but may contain more than one non-tumor cells. The fraction that is detected as positive for containing cancer-only marker sequences should contain a CTC. By counting the number of positive fractions, the number of CTCs in a sample can be determined. The actual number of CTCs in the sample can, if needed, be further calculated using Poisson statistics modeling (Lievens A, et al. PLoS One. 2016; 11(5):e0153317). Because of low background signals from surrounding blood cells, using cancer-only markers allows detection of a single CTC in the presence of thousands of blood cells. In this scenario, the method may be used to detect CTCs in minimally processed or non-enriched blood cell samples. In another embodiment, CTC-enriched cells are fractionated such that each fraction practically contains no more than one cell. For example, 1000 cells are fractionated into a 20000-well dPCR mini-chip so that most fraction will contain no more than one single cell. Or cells can be encapsulated into microdroplets in such a configuration that most microdroplets will contain one cell at most. Each fraction contains either one CTC, one non-tumor cell, or no cell. Smaller difference in gene expression (e.g. 2 fold difference) can be differentiated in this scenario since the comparison is directly made between one CTC cell and a non-tumor cell. In a single CTC without interference from other cells, it is possible to detect gene number variation within the cell.

The PCR products in each fraction can be detected using any conventional method to detect nucleic acids that are well known in the art. The methods for detection of nucleic acids include, for example, using a non-specific dsDNA binding dye (e.g. SYBR Green) or a sequence-specific reporter probe (e.g. Taqman® Probe). The PCR products can be detected at the end of the reaction or during the PCR cycles. The tumor-specific sequences can be detected by digital PCR which detects the PCR products at the end of the PCR cycle. The tumor-specific sequences can also be detected by real time quantitative PCR which monitors the amplification curve during the PCR cycles. The PCR amplification curve can provide additional information to aid in evaluating true and false positives in the PCR. In a preferred embodiment, the amplified tumor-specific sequences are detected by a real time quantitative PCR or digital PCR using a sequence-specific reporter probe. The sequence-specific reporter probe is designed to comprise a signature sequence complementary to a sequence on the amplicon of a tumor-specific sequence. Different sequence-specific reporter probes can be conjugated to the same fluorophore and are used to detect the presence of any sequence of a group of tumor-specific sequences. On the other hand, different sequence-specific reporter probes can be conjugated to different fluorophores. For example, cancer genes related to different cancer types can be detected by different fluorophores, allowing determination of cancer linage of a CTC. Tumor-specific sequences related to RNA transcripts and ones related to genomic DNA mutations can also be distinguished by use of different fluorophores. To count the number of CTCs in a sample, the amount of tumor-specific sequences is calculate for every fraction. The fractions can be divided into three groups, a no cell group which has no signal, a non-tumor cell group which has low background signals, and a CTC group which has signals significantly higher than the background signal. The number of CTCs is the number of fractions in the CTC group.

Although the method is described here as counting circulating tumor cells in blood or lymph cell samples, the method can be similarly applied to count rare occurrence of a specific type of cells in the context of other background cells if the specific type of cells have unique distinguishable marker gene(s). For example, it can be used to count virus-infected cells in a population of cells by detecting the presence of virus genes.

It is reported that CTCs can be detected in the early phase of cancers. In lieu of using tissue biopsy for cancer diagnostics, counting CTCs in the peripheral blood can be used as an noninvasive and sensitive method to detect the presence of a cancer. In one embodiment, the present invention provides a method of detecting the presence of cancer in a subject, comprising: a) determining the presence of CTCs in a blood sample or a lymphatic fluid sample from the subject using the method described herein; and b) using the presence of CTCs as indicative of the presence of cancer in the subject. The presence of at least 1, 2, 3, 4, or 5 CTCs in certain amount of blood sample can be used as a criteria for diagnosing a cancer. The pan-cancer specific marker sequences can be used to determine the presence of a cancer, or a marker sequence set of a specific cancer type can be used to determine if a subject has a specific type of cancer. To increase the specificity of detection of a CTC in a clinical sample, more than one marker sequences can be used as the criteria of positive detection. For example, the presence of more than one pan-cancer specific marker sequences can be used for detection of a positive CTC. For a subject susceptive of a particular type of cancer, the marker sequences specific for the particular cancer type plus the one or more pan-cancer specific marker sequences can be chosen for detection of CTCs.

In some embodiment, the present invention provides a method of making prognostic prediction of a cancer treatment in a patient, comprising a) counting CTCs in a patient's blood sample or lymphatic fluid sample at different times during or after the cancer treatment using the method described herein; and b) using a increasing and decreasing count of CTCs in patient's sample as indicative of a poor and good prognosis, respectively. In some embodiment, the CTCs are tumor cells that express metastasis-related cancer genes.

In some embodiment, the present invention provides a method of detecting cancer metastasis in a patient, comprising a) detecting the presence of CTCs with expression of metastasis-related cancer genes in patient's blood sample or lymphatic fluid sample using the method described herein; and b) using the presence of CTCs with expression of metastasis-related cancer genes as indicative of the presence of cancer metastasis in the patient.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

What is claimed is:

1. A method for counting circulating tumor cells (CTCs) in a cell sample, comprising the steps of:
a) fractionating the cell sample into a plurality of equal fractions, wherein more than 50% of the fractions contain no more than one CTC per fraction;
b) extracting RNA and generating cDNA from the extracted RNA, and/or extracting DNA from cells in each fraction;
c) performing amplification reactions in each fraction using at least one pair of tumor-specific primers for amplification of tumor-specific sequences;
d) detecting amplified tumor-specific sequences in each fraction; and
e) counting the number of fractions with amplified tumor-specific sequences to determine the number of CTCs in the sample.

2. The method of claim 1, wherein each fraction contains no more than one CTC.

3. The method of claim 1, wherein the cell sample is enriched or not enriched for CTCs.

4. The method of claim 1, wherein more than 50% of the fractions contain no more than one cell per fraction.

5. The method of claim 1, wherein the cell sample is a blood sample or a lymph sample.

6. The method of claim 1, wherein the cell sample is a blood sample deprived of red blood cells and white blood cells.

7. The method of claim 1, wherein the tumor-specific sequences are RNA transcripts that have higher expression levels in tumor cells than normal blood cells or lymphocytes, preferably have no detectable expression in normal blood cells or lymphocytes.

8. The method of claim 1, wherein the tumor-specific primers are designed to amplify cDNA molecules of cancer genes selectively expressed in one or more types of cancer cells.

9. The method of claim 8, the cancer types are selected from the group consisting of breast cancer, lung cancer, liver cancer, pancreatic cancer, prostate cancer, stomach cancer, kidney cancer, intestinal cancer, colon cancer and melanoma.

10. The method of claim 1, wherein the tumor-specific primers are designed to amplify cDNA molecules of cancer genes related to metastasis.

11. The method of claim 1, wherein the cell sample is fractionated into more than 10, 100, 1000, 10,000 or 100,000 fractions.

12. The method of claim 1, wherein the tumor-specific sequences are amplified and detected by quantitative PCR or digital PCR.

13. The method of claim 1, wherein the tumor-specific sequences are detected by sequence-specific reporter probes.

14. The method of claim 1, wherein the tumor-specific sequences selective for different cancer types are detected by different reporter probes.

15. The method of claim 1, wherein the tumor-specific sequence is a genomic DNA sequence containing a tumor-specific mutation.

16. The method of claim 1, wherein the tumor-specific sequence is a cancer-related gene that undergoes copy number multiplication in tumor cells.

17. A method of detecting the presence of cancer in a subject, comprising
a) determining the presence of CTCs in a blood sample or a lymphatic fluid sample from the subject using the method of claim 1; and
b) using the presence of CTCs as indicative of the presence of cancer in the subject.

18. A method of making prognostic prediction of a cancer treatment in a patient, comprising
a) counting CTCs in a patient's blood sample or lymphatic fluid sample at different times during or after the cancer treatment using the method of claim 1; and
b) using a increasing and decreasing count of CTCs in patient's sample as indicative of a poor and good prognosis, respectively.

19. The method of claim 18, wherein the CTCs are tumor cells that express metastasis-related cancer genes.

20. A method of detecting cancer metastasis in a patient, comprising
a) detecting the presence of CTCs with expression of metastasis-related cancer genes in patient's blood sample or lymphatic fluid sample using the method of claim 1; and
b) using the presence of CTCs with expression of metastasis-related cancer genes as indicative of the presence of cancer metastasis in the patient.
